(19) United States
(12) Patent Application Publication
(10) Pub. No.: US 2023/0191412 A1
(43) Pub. Date: Jun. 22, 2023

(54) Two-Stage Magnetic Device for Sorting Biological Objects

(71) Applicant: Applied Cells Inc., Santa Clara, CA (US)

(72) Inventors: Yuchen Zhou, San Jose, CA (US); Bing K. Yen, Cupertino, CA (US)

(21) Appl. No.: 18/111,486

(22) Filed: Feb. 17, 2023

Related U.S. Application Data
(63) Continuation-in-part of application No. 18/072,362, filed on Nov. 30, 2022.
Application No. 18/072,362 is a continuation-in-part of application No. 16/729,398, filed on Dec. 29, 2019, now Pat. No. 11,571,696.
Application No. 16/729,398 is a continuation-in-part of application No. 15/911,115, filed on Mar. 3, 2018.
(60) Provisional application No. 63/406,437, filed on Sep. 14, 2022.

(51) Int. Cl. B01L 3/00; H01F 7/02; C12N 13/00

(52) U.S. Cl. CPC B01L 3/502761; H01F 7/02; C12N 13/00; B01L 2400/043; B01L 2200/0668; B01L 2400/086

(57) ABSTRACT

The present invention is directed to a method for sorting biological objects including the steps of providing a magnetic device that includes a conduit or channel having upstream and downstream sections and a magnetic means for generating first and second magnetic fields in the upstream and downstream sections, respectively; flowing a sample fluid that includes magnetically labeled biological objects and unlabeled biological objects through the upstream section to magnetically saturate the magnetically labeled biological objects by the first magnetic field; and flowing the sample fluid from the upstream section continuously to the downstream section to collect the magnetically labeled biological objects on a wall of the downstream section by the second magnetic field, wherein the first magnetic field in the upstream section has a higher average field strength than the second magnetic field in the downstream section.

### CROSS-REFERENCE TO RELATED APPLICATIONS

The present application is a continuation-in-part of application Ser. No. 18/072,362, filed on Nov. 30, 2022, which claims priority to provisional application No. 63/406,437, filed on Sep. 14, 2022, and is a continuation-in-part of application Ser. No. 16/729,398, filed on Dec. 29, 2019, which is a continuation-in-part of application Ser. No. 15/911,115, filed on Mar. 3, 2018. All of these applications are incorporated herein by reference in their entirety, including their specifications.

### BACKGROUND

The present invention relates to a method and device for sorting biological objects, and more particularly, to embodiments of a two-stage magnetic device for sorting magnetic or magnetically labeled biological objects in a fluid.

The separation and sorting of biological objects or cells is critical to various biomedical applications, such as diagnostics and therapeutics. Biological objects may be sorted based on their respective physical properties, such as size and density, and biochemical properties, such as surface antigen expression.

In a biological object sorting process effectuated by an applied magnetic field, the biological object, such as a cell, which is typically nonmagnetic, can be magnetized for magnetic sorting purpose by attaching antibody-conjugated magnetic beads thereto, a process commonly known as magnetic labeling. FIG. 1A shows a cell 50 including a plurality of surface markers or antigens 52 on the cell surface thereof, and a plurality of antibody-conjugated magnetic beads 54 suspended in a fluid. Each of the antibody-conjugated magnetic beads 54 includes a magnetic entity 56 conjugated with one or more antibodies or other ligands 58, such as peptides and aptamers, that correspond to the surface markers 52. After an incubation period, the magnetic beads 54 may be directly attached to the cell 50 via the antigen-antibody interaction to form a magnetically labeled cell as shown in FIG. 1B, in a process known as direct labeling.

Alternatively, magnetic beads may be attached to a cell through an indirect labeling process. FIG. 2A shows a cell 50 including a plurality of surface markers or antigens 52 on the cell surface thereof, a plurality of intermediary links 60, and a plurality of magnetic beads 62 suspended in a fluid. Each of the intermediary links 60 includes one or more linking molecules 64, such as biotin or phycoerythrin (PE), conjugated to a primary antibody 66 that corresponds to the surface markers 52 of the cell 50. Each of the magnetic beads 62 includes a magnetic entity 56 conjugated with one or more secondary antibodies or ligands 68, such as streptavidin, that target the linking molecules 64. After an incubation period, the intermediary links 60 may attach to the cell 50 via the antigen-antibody interaction, and the magnetic beads 62 may further attach to the intermediary links 60 via PE-antibody, biotin-streptavidin, or other types of interactions, thereby forming a magnetically labeled cell as shown in FIG. 2B.

The magnetic beads 54 and 62 should ideally exhibit no magnetic moment in the absence of an applied magnetic field, thereby making the labeled cells indistinguishable from other biological objects in a cell suspension. As such, the magnetic entity 56 of the magnetic beads 54 and 62 normally consists of a magnetic nanoparticle or an aggregate of magnetic nanoparticles encapsulated in a nonmagnetic matrix because a magnetic particle, ferromagnetic (e.g., iron) or ferrimagnetic (e.g., iron oxide), may exhibit superparamagnetism as its size is reduced to tens of nanometers. A superparamagnetic particle behaves like a nonmagnetic particle in the absence of an external magnetic field but becomes magnetic when exposed to a magnetic field. During a cell sorting process, the magnetic nanoparticles of the magnetically labeled cells are first magnetized by sufficiently high magnetic field generated by a magnetic separator device and then attracted to regions of high magnetic field gradient.

After cells in sample fluid are magnetically labeled, they can be sorted or separated from the other non-labeled cells or biological objects in the sample fluid by a magnetic separator device. FIG. 3A shows a conventional magnetic separator device 70 comprising a container vessel 72 for holding static sample fluid 74 that contains the magnetically labeled cells 76 and a permanent magnet 78 placed in close proximity to a wall of the container vessel 72. The permanent magnet 78 generates a magnetic field in the container vessel 72 with the magnetic field gradient pointing towards the permanent magnet 78. After sufficient time, the magnetically labeled cells 76 will be gradually pulled by the force produced by the magnetic field towards the vessel wall and form an aggregate at the vessel wall, as shown in FIG. 3B. Because the magnetic field strength rapidly decreases as the distance from the permanent magnet 78 increases, the size of the vessel 72 and the sample fluid volume will be adversely limited.

FIG. 4 illustrates another conventional magnetic separator device 80 that separates magnetically labeled cells in static sample fluid contained in one or more wells 82. The magnetic device 80 uses multiple ferromagnetic poles 84, each of which has a trapezoidal tip, to act as a guide to concentrate the magnetic flux generated by multiple permanent magnets 86 attached thereto to increase the magnetic field strength and gradient near their tips. The corresponding magnetic field distribution, as delineated by magnetic field lines 88, shows that the magnetic field is strongest between the side surfaces of adjacent trapezoidal tips, as indicated by the small spacing between the field lines 88. By contrast, the magnetic field is much weaker above the pole tips, as indicated by the large spacing between the field lines 88. Accordingly, this necessitates the bottom portion of each well 82 to be disposed between the side surfaces of the pole tips, where the magnetic field is strong. The magnetically labeled cells in the conical-shaped wells 82 will be collected or condensed on or near the bottom of the wells 82 adjacent to the side surfaces of the trapezoidal tips of the ferromagnetic poles 84. Compared with the magnetic separator device 70 utilizing only the permanent magnet 78, the magnetic separator device 80 may improve the magnetic field strength and gradient by using the ferromagnetic poles 84 to concentrate the magnetic flux. Both devices 70 and 80, however, are designed to treat static sample fluid and may thus have limited throughput.

FIG. 5A illustrates a conventional magnetic separator device 90 that separates the magnetically labeled cells 76 as the sample fluid flows through the device 90. The device 90 includes a conduit 92 disposed between a pair of permanent magnets 94 that generate a magnetic field 96 across the conduit 92. The conduit 92 is filled with a column of porous aggregate of ferromagnetic or ferrimagnetic particles or spheres 98 that may be magnetized by the magnetic field 96 and produce relatively strong localized magnetic field and field gradient in small gaps between the particles or spheres 98, thereby magnetizing the magnetically labeled cells 76 and attracting them to the surface of the particles or spheres 98. Compared with the magnetic beads attached to the magnetically labeled cells 76, the ferromagnetic or ferrimagnetic particles or spheres 98 are much larger and may produce remanent magnetization after the permanent magnets 94 are removed from the conduit 92. The remanent magnetization may prevent or hinder the detachment of the magnetically labeled cells 76 from the surface of the particles or spheres 98 even after the removal of the magnetic field 96. While the magnetic separator device 90 may operate in a continuous flow manner and thus may have potentially a higher throughput than the magnetic separators 70 and 80 that operate in a static manner, the recovery of the magnetically labeled cells in certain applications (e.g., positive selection process where the magnetically labeled cells are the target cells) may be lower without vigorously flushing the conduit 92 to dislodge the magnetically labeled cells 76 from the surface of the particles or spheres 98.

The column of porous aggregate of soft magnetic particles or spheres 98 in the conduit 92 may be replaced by one or more meshes 102 made of a ferromagnetic or ferrimagnetic material as shown in FIG. 5B. The magnetic separator device 100 may reduce the remanent magnetization encountered in the device 90 because the wires in mesh 102 have smaller dimensions than the ferromagnetic or ferrimagnetic particles or spheres 98. However, the larger opening between adjacent wires in the mesh 102 may also weaken the localized magnetic field, thereby decreasing the device throughput. Both column-based devices 90 and 100 may introduced unwanted contaminants into the sample fluid as it flows through the ferromagnetic or ferrimagnetic material in the conduit 92.

FIG. 6 shows another magnetic separator device 104, which operates in a continuous flow manner without using a column of porous aggregate of ferromagnetic or ferrimagnetic material, thereby obviating the potential contamination and recovery issues. The column-free device 104 includes a conduit 106 surrounded by a radial array of ferromagnetic poles 108 that conduct magnetic flux from a plurality of permanent magnets 110 and 112. The sample fluid flows through the conduit 106 along a direction perpendicular to the figure. The magnetic separator device 104 essentially rearranges the linear array of the ferromagnetic poles 84 of the static magnetic separator device 80 in a radial manner to create a magnetic periodic field in the center of the radially arranged ferromagnetic poles 108 and permanent magnets 110 and 112. Like the static device 80 shown in FIG. 4, the corresponding magnetic field distribution generated by the device 104, as delineated by magnetic field lines 114 between the trapezoidal tips of the ferromagnetic poles 108, shows that the magnetic field is strongest between the side surfaces of adjacent trapezoidal tips, as indicated by the small spacing between the field lines 114, and much weaker above the pole tips (i.e., inside the conduit 106), as indicated by the large spacing between the field lines 114. However, unlike the wells 82 that extend into the regions between the side surfaces of two adjacent trapezoidal tips, the conduit 106 of the magnetic separator device 104 does not extend into such regions, thereby making the magnetic field in the conduit 106 considerably weaker. This is further exacerbated by the limited time exposed to the magnetic field as the sample fluid flows through the conduit 106.

For the foregoing reasons, there is a need for a magnetic separator device and method that can rapidly separate or sort magnetically labeled cells without introducing potential contaminants into the sample.

### SUMMARY

The present invention is directed to a method that satisfies this need. A method having features of the present invention for sorting biological objects includes the steps of providing a magnetic device that includes a conduit or channel having upstream and downstream sections and a magnetic means for generating first and second magnetic fields in the upstream and downstream sections, respectively; flowing a sample fluid that includes magnetically labeled biological objects and unlabeled biological objects through the upstream section to magnetically saturate the magnetically labeled biological objects by the first magnetic field; and flowing the sample fluid from the upstream section continuously to the downstream section to collect the magnetically labeled biological objects on a wall of the downstream section by the second magnetic field, wherein the first magnetic field in the upstream section has a higher average field strength than the second magnetic field in the downstream section.

According to another aspect of the present invention, a magnetic device having features of the present invention for sorting biological objects includes a first magnetic assembly, a second magnetic assembly disposed adjacent to the first magnetic assembly, and a conduit. The first magnetic assembly includes a first magnetic flux source; a first magnetic flux guide having a first base and a first tip with a first blunt end; and a second magnetic flux guide having a second base and a second tip with a second blunt end facing the first tip, wherein the first and second bases are magnetically coupled to the first magnetic flux source to generate opposite magnetic polarities on the first and second tips, respectively, thereby producing a first magnetic field between the first and second tips. The second magnetic assembly includes a second magnetic flux source; a third magnetic flux guide having a third base and a third tip with a third blunt end; and a fourth magnetic flux guide having a fourth base and a fourth tip with a substantially pointed end facing the third tip, wherein the third and fourth bases are magnetically coupled to the second magnetic flux source to generate opposite magnetic polarities on the third and fourth tips, respectively, thereby producing a second magnetic field between the third and fourth tips. The conduit is operably disposed between the first blunt end of the first tip and the second blunt end of the second tip and between the third blunt end of the third tip and the substantially pointed end of the fourth tip.

### BRIEF DESCRIPTION OF THE DRAWINGS

These and other features, aspects, and advantages of the present invention will become better understood with regard to the following description, appended claims, and accompanying drawings where:

FIGS. 1A and 1B illustrate formation of a magnetically labeled cell by direct labeling process;

FIGS. 2A and 2B illustrate formation of a magnetically labeled cell by indirect labeling process;

FIGS. 3A and 3B illustrate sorting of magnetically labeled cells by a conventional static magnetic separator device;

FIG. 4 illustrates another conventional magnetic separator device for sorting magnetically labeled cells in static sample fluid;

FIGS. 5A and 5B illustrate two conventional magnetic separator devices that utilize a conduit filled with a column of ferromagnetic or ferrimagnetic objects for sorting magnetically labeled cells flowing through the column;

FIG. 6 is a cross-sectional view corresponding to a magnetic separator device for sorting magnetically labeled cells flowing through a conduit;

FIG. 7 is a plot illustrating dynamic magnetization response to rapidly changing magnetic field for a magnetically labeled cell having a finite relaxation time;

FIG. 8 illustrates a flow-through magnetic separator device including two stages of magnetic fields for sorting magnetically labeled cells in accordance with an embodiment of the present invention;

FIG. 9 is a cross-sectional view of a first magnetic assembly of a magnetic separator device for generating a first magnetic field in an upstream section of a conduit in accordance with an embodiment of the present invention;

FIG. 10 is a cross-sectional view of a first magnetic assembly of a magnetic separator device for generating a first magnetic field in an upstream section of a conduit in accordance with another embodiment of the present invention;

FIG. 11 is a cross-sectional view of a first magnetic assembly of a magnetic separator device for generating a first magnetic field in an upstream section of a conduit in accordance with still another embodiment of the present invention;

FIG. 12 is a cross-sectional view of a first magnetic assembly of a magnetic separator device for generating a first magnetic field in an upstream section of a conduit in accordance with yet another embodiment of the present invention;

FIG. 13 is a cross-sectional view of a second magnetic assembly of a magnetic separator device for generating a second magnetic field in a downstream section of a conduit in accordance with an embodiment of the present invention;

FIG. 14 is a cross-sectional view of a second magnetic assembly of a magnetic separator device for generating a second magnetic field in a downstream section of a conduit in accordance with another embodiment of the present invention;

FIG. 15 is a cross-sectional view of a second magnetic assembly of a magnetic separator device for generating a second magnetic field in a downstream section of a conduit in accordance with still another embodiment of the present invention; and

FIG. 16 is a cross-sectional view of a second magnetic assembly of a magnetic separator device for generating a second magnetic field in a downstream section of a conduit in accordance with yet another embodiment of the present invention.

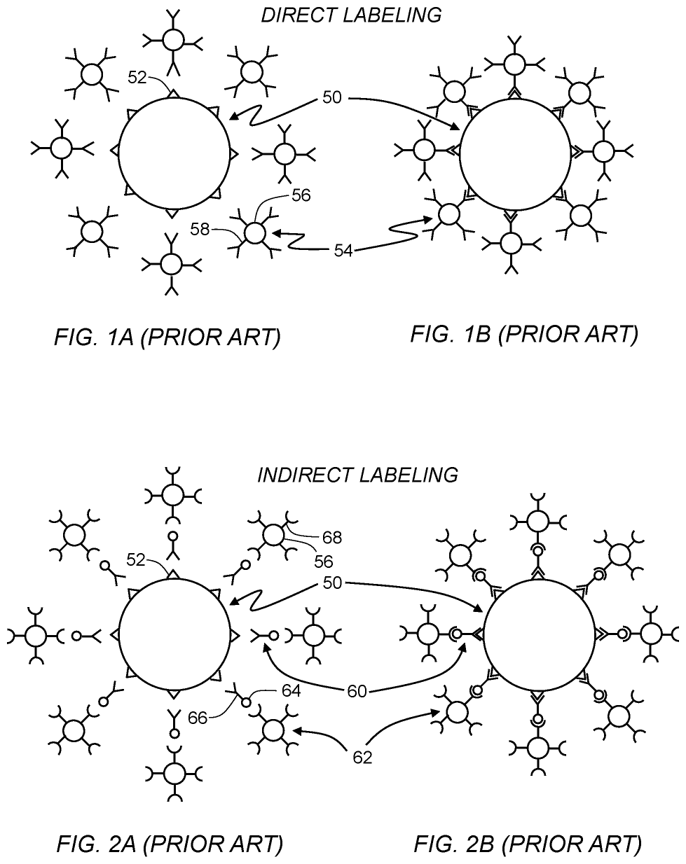

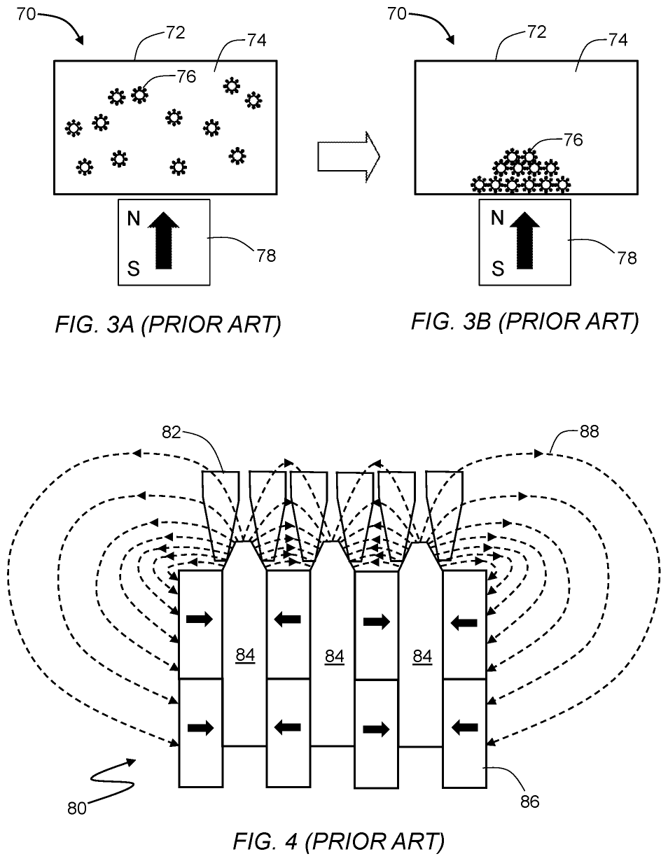

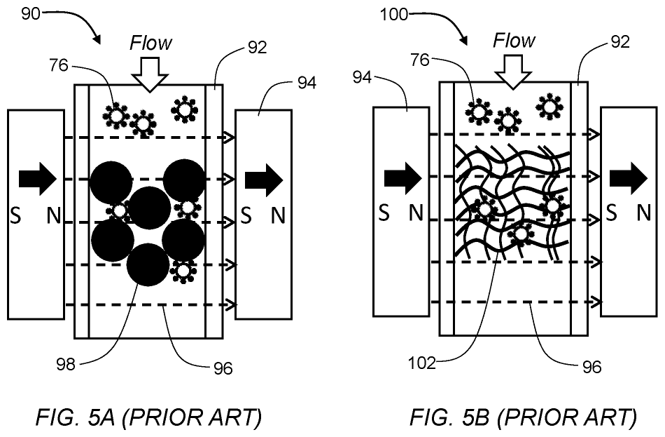

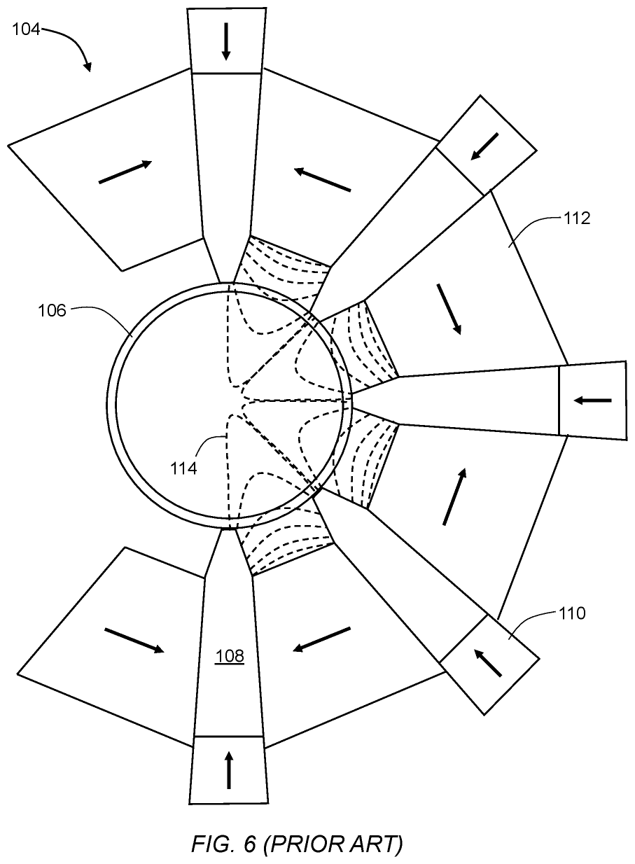

For purposes of clarity and brevity, like elements and components will bear the same designations and numbering throughout the Figures, which are not necessarily drawn to scale.

### DETAILED DESCRIPTION

In the Summary above and in the Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously, except where the context excludes that possibility, and the method can include one or more other steps which are carried out before any of the defined steps, between two of the defined steps, or after all the defined steps, except where the context excludes that possibility.

The term “biological objects” may be used herein to include cells, bacteria, viruses, molecules, particles including RNA and DNA, cell cluster, bacteria cluster, molecule cluster, and particle cluster.

The term “biological sample” may be used herein to include blood, body fluid, tissue extracted from any part of the body, bone marrow, hair, nail, bone, tooth, liquid and solid from bodily discharge, or surface swab from any part of body. “Fluid sample,” or “sample fluid,” or “liquid sample,” or “sample solution” may include a biological sample in its original liquid form, biological objects being dissolved or dispersed in a buffer liquid, or a biological sample dissociated from its original non-liquid form and dispersed in a buffer fluid. A buffer fluid is a liquid into which biological objects may be dissolved or dispersed without introducing contaminants or unwanted biological objects. Biological objects and biological sample may be obtained from human or animal. Biological objects may also be obtained from plants and environment including air, water, and soil. A fluid sample may contain various types of magnetic or optical labels, or one or more chemical reagents that may be added during various process steps.

The term “sample flow rate” or “flow rate” may be used herein to represent the volume amount of a fluid sample flowing through a cross section of a channel, or a conduit, or a fluidic part, or a fluidic path in a unit time.

The term “relative fraction” may be used herein to represent the ratio of a given quantity of biological objects or particles to the total quantity of all biological objects or particles present in a fluid sample.

In the art of cell sorting or enrichment, the target population of biological objects is referred to as the “specific” objects of interest and those biological objects that are isolated, but are not desired, are termed “non-specific.” The term “purity” describes the concentration or relative fraction of target or specific biological objects of interest and is quantified by the number of target biological objects divided by the total number of biological objects expressed in percentage. The term “recovery ratio” describes the sorting efficiency of biological objects and is quantified by the number of target biological objects recovered after sorting divided by the number of target biological objects present in the initial sample expressed in percentage.

In the magnetic sorting process, the magnetically labeled biological objects or cells are first magnetized by sufficiently high magnetic field and then attracted to regions of high magnetic field gradient. However, in designing a flow-through magnetic separator device for cell sorting, it is often a compromise between high magnetic field strength for magnetizing as many cells as possible and high magnetic field gradient for attracting or moving magnetized cells as fast as possible, because devices with high magnetic field strength and devices with high magnetic field gradient may have conflicting attributes.

It is generally accepted that the individual nanoparticles used in the magnetic beads 54 and 62 magnetize and demagnetize reversibly with applied magnetic field as measured quasi-statically. However, this reversible magnetization and demagnetization behavior, which is an attribute of superparamagnetism, may not be valid under a dynamic condition, where rapidly changing magnetic field may cause a latency in the material response, especially for larger nanoparticles with inherently longer relaxation time (for example, see U.S. Pat. No. 8,114,683). Furthermore, the aggregation of superparamagnetic particles in a magnetic bead may increase the relaxation time of the magnetic aggregate as a whole if the particles are closely packed in the magnetic bead, thereby causing a latency in the material response when demagnetize under the dynamic condition. Moreover, the further aggregation of multiple magnetic beads on a cell surface may also further increase the relaxation time of the magnetic aggregate as a whole. Accordingly, any of the circumstances discussed above may cause a magnetically labeled biological object or cell with a finite relaxation time not to behave like an ideal superparamagnetic particle.

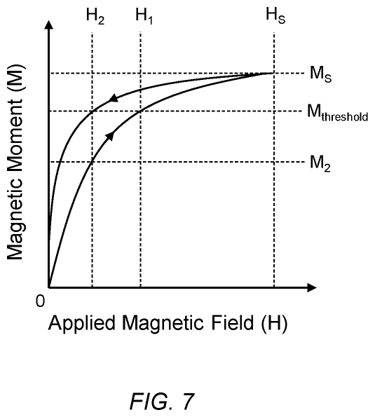

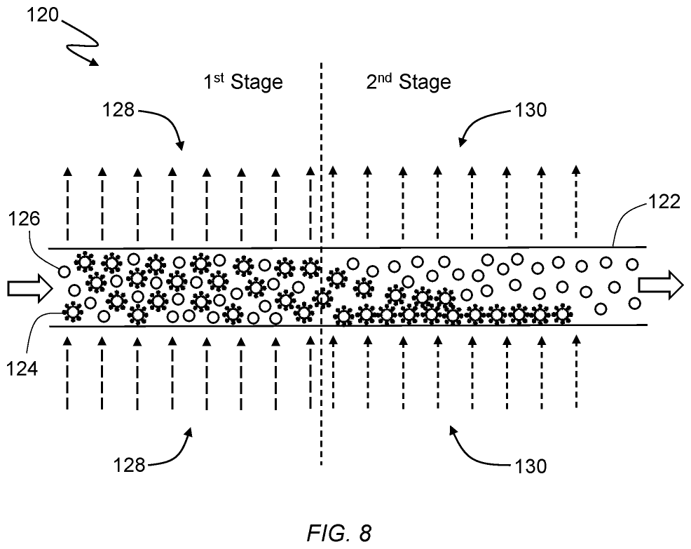

Without being bound to any theory, it is possible that a magnetically labeled biological object or cell with a finite relaxation time may exhibit a magnetic hysteresis behavior characterized by a magnetic moment vs. applied magnetic field plot shown in FIG. 7 under a dynamic condition involving rapidly changing magnetic field. Accordingly, the inventors disclose herein a two-stage, column-free, flow-through magnetic separator device that can generate both high magnetic field strength to magnetize cells and high magnetic field gradient to attract or move cells.

The hysteresis loop in FIG. 7 illustrates the magnetic moment (M) or the degree of magnetization of a magnetically labeled cell as the applied magnetic field (H) increases from zero to the saturation field (H_{S}) and back to zero. The magnetic moment (M) of the magnetically labeled cell increases with the applied magnetic field (H) until the moment asymptotically approaches the saturation moment (M_{S}) at or near the saturation field (H_{S}), beyond which the moment remains relatively constant. After the magnetization of the magnetically labeled cell is saturated at Hs, the moment decreases with decreasing field but at a more gradual rate compared to the moment increase owing to the latency caused by the rapidly decreasing magnetic field. For example and without limitation, assuming a magnetically labeled cell needs to be magnetized above M_{threshold} to generate enough force for cell sorting, and applied magnetcic field greater than H₁ would be needed. However, after the magnetization of the magnetically labeled cell is first saturated, a magnetic field as low as H₂, which may be significantly lower than Hi, may be used to maintain the magnetization above M_{threhold}.

Accordingly, it is conceived by the inventors that a flow-through magnetic separator device 120 includes a channel or conduit 122 exposing magnetically labeled biological objects or cells 124 and other unlabeled biological objects or cells 126 flowing therethrough to two different magnetic fields 128 and 130 at different stages as shown in FIG. 8. The first magnetic field 128 generated at the upstream or first stage may be used to magnetically saturate as many magnetically labeled cells 124 as possible as the mixture of the magnetically labeled cells 124 and the unlabeled biological objects 126 flows through the conduit 122. At the first stage, the magnetic separator device 120 may generate sufficiently high magnetic field strength over the cross section of the conduit 122 to magnetically saturate as many magnetically labeled cells 124 as possible. However, at this stage, the magnetic separator device 120 may not generate sufficient magnetic field gradient to attract the magnetized magnetically labeled cells 124 to collect them on the inner wall surface of the conduit 122. Therefore, both the magnetized magnetically labeled cells 124 and unlabeled biological objects 126 may continuously flow through the conduit 122 at the upstream or first stage.

After the magnetically labeled cells 124 are magnetically saturated at the first stage by the first magnetic field 128, the mixture of the magnetically labeled cells 124 and the unlabeled biological objects 126 flows uninterruptedly to the downstream or second stage, where the magnetic separator device 120 may generate a second magnetic field 130 that has sufficiently high magnetic field gradient to attract the magnetized magnetically labeled cells 124 to collect them on the inner wall surface of the conduit 122 to form one or more conglomerates or aggregates, thereby separating them from the unlabeled biological objects 126 that continuously flow through the conduit 122. Prioritizing high magnetic field gradient to attract the magnetized magnetically labeled cells 124, the magnetic field strength over most of the cross section of the conduit 122 at the second stage may be lower than that of the first stage. However, the lower magnetic field strength of the second magnetic field 130 may be sufficient to maintain the magnetization of the magnetically labeled cells 124 at or above a threshold level for the cells 124 to condense or accumulate on the conduit surface because the magnetically labeled cells 124 had previously been magnetically saturated at the first stage by the first magnetic field 128.

For example and without limitation, referring back to FIG. 7, a magnetic bead may be magnetically saturated at a magnetic field of Hs or higher to gain a magnetic moment of Ms at the first stage and then continuously move to the second stage, where the magnetic field gradient may be higher while the magnetic field strength may be lower (e.g., H₂). The magnetic moment corresponding to H₂ would be M_{threshold} at the second stage as the field decreases from Hs to H2. However, if the magnetic particle were to enter the second stage directly, bypassing the first stage without being previously saturated, then the magnetic moment corresponding to H₂ would simply be M₂, which may be significantly lower than M_{threshold}. Also, if the transition from the first magnetic field at the first stage to the second magnetic field at the second stage is interrupted, causing the magnetic field to temporarily drop to zero, then the magnetic moment corresponding to H₂ at the second stage would also be M₂, as the field increases from zero to H₂.

Therefore, the use of the first magnetic field 128 in the first stage to magnetically saturate the magnetically labeled cells 124 essentially boosts the magnetization of the same cells 124 at the second stage, where the magnetic field gradient may be prioritized over the magnetic field strength in the conduit 122 to extract the magnetically labeled cells 124 from the sample fluid. Compared with conventional magnetic separator devices having only single stage magnetic field that is usually a compromise between magnetic field strength and magnetic field gradient, the present invention advantageously provides enhanced magnetization of the magnetically labeled cells without compromising the high magnetic field gradient for extracting the same cells from the sample fluid.

According to an embodiment of the present invention, a method for sorting biological objects begins by providing a magnetic device 120 that includes a conduit 122 having an upstream (i.e., first stage) section and a downstream (i.e., second stage) section and a magnetic means for generating first and second magnetic fields 128 and 130 in the upstream and downstream sections, respectively. The process continues by flowing a sample fluid that includes magnetically labeled biological objects 124 and unlabeled biological objects 126 through the upstream section of the conduit 122 to magnetically saturate the magnetically labeled biological objects 124 by the first magnetic field 128; and flowing the sample fluid from the upstream section continuously to the downstream section of the conduit 122 to collect the magnetically labeled biological objects 124 on an inner wall surface of the downstream section of the conduit 122 by the second magnetic field 130. The second magnetic field 130 may have a higher field gradient than the first magnetic field 128 to attract or move the magnetized magnetically labeled biological objects 124 to the inner wall surface of the conduit 122. Higher field gradient may correspond to a rapid drop in the magnetic field strength. Accordingly, the second magnetic field 130 may have a lower average field strength than the first magnetic field 128.

According to another embodiment of the present invention, a two-stage magnetic separator device 120 for sorting biological objects includes a conduit 122 having an upstream (i.e., first stage) section and a downstream (i.e., second stage) section and a magnetic means for generating the first and second magnetic fields 128 and 130 in the upstream and downstream sections, respectively. The first magnetic field 128 may have a higher average field strength than the second magnetic field 130. The second magnetic field 130 may have a higher field gradient than the first magnetic field 128. The magnetic means may include a first magnetic assembly for generating the first magnetic field 128 in the upstream section of the conduit 122 and a second magnetic assembly for generating the second magnetic field 130 in the downstream section of the conduit 122. The first magnetic assembly may include a magnetic flux source, which may comprise one or more permanent magnets and/or one or more electric magnets, and a set of magnetic flux guides for guiding the magnetic flux and/or shaping the first magnetic field 128. The second magnetic assembly is disposed adjacent to the first magnetic assembly and may be physically attached thereto. The second magnetic assembly may include another magnetic flux source, which may comprise one or more permanent magnets and/or one or more electric magnets, and another set magnetic flux guides for guiding the magnetic flux and/or shaping the second magnetic field 130.

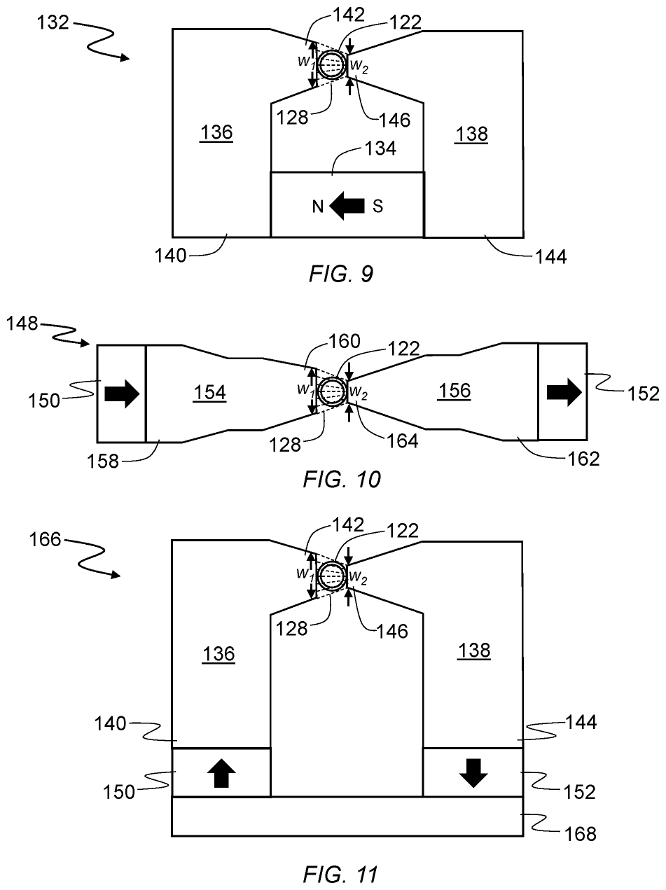

FIG. 9 is a cross-sectional view of a first magnetic assembly 132 in accordance with an embodiment of the present invention. The first magnetic assembly 132, which generates a first magnetic field 128 in the upstream section of the conduit 122, includes a magnetic flux source, which comprises a permanent magnet 134, and first and second magnetic flux guides 136 and 138 for conducting the magnetic flux and shaping the first magnetic field 128. The conduit 122, the permanent magnet 134, and the first and second magnetic flux guides 136 and 138 extend in a direction perpendicular to the drawing.

The first magnetic flux guide 136 has a first base 140 that may collect magnetic flux and a first tip 142 that may emit magnetic field. The second magnetic flux guide 138 has a second base 144 that may collect magnetic flux and a second tip 146 that may emit magnetic field. The first and second magnetic flux guides 136 and 138 are bent with their tips 142 and 146 pointed toward each other. The first and second bases 140 and 144 are physically and/or magnetically coupled to the permanent magnet 134 at its two poles (e.g., North and South poles), respectively, for conducting the magnetic flux from the permanent magnet 134, thereby generating opposite magnetic polarities on the first and second tips 142 and 146, respectively. The first magnetic flux guide 136 has a smaller cross section, which may be defined herein as the cross-sectional area perpendicular to the flux flow, at the first tip 142 than the first base 140 in order to concentrate the magnetic flux collected from the permanent magnet 134 to the first tip 142. Similarly, the second tip 146 has a smaller cross section than the second base 144 to concentrate the magnetic flux collected from the permanent magnet 134 to the second tip 146. Accordingly, each of the tips 142 and 146 has a higher magnetic flux density than the corresponding base 140 or 144. The first and second tips 142 and 146 emit and shape the first magnetic field 128 in the gap therebetween from the concentrated magnetic flux.

The first magnetic field 128 is shaped by the tip geometry of the magnetic flux guides 136 and 138. Each of the first and second tips 142 and 146 may have a tapering shape with a blunt or flat end. The flat ends of the first and second tips 142 and 146 may be substantially parallel to each other with the conduit 122 operably disposed between the two flat ends. The conduit 122 may be in contact with or in close proximity to the first and second flat ends during the cell sorting process. The width of one tip end (e.g., the first tip 142, w₁) may be wider than the inner diameter of the conduit 122 while the width of the other tip end (e.g., the second tip 146, w₂) may be comparable to or smaller than the inner diameter of the conduit 122. This tip end geometric configurations may expose most or all of the interior of the conduit 122 to a magnetic field with high field strength provided that the base-to-tip cross section ratios (i.e., flux concentration factor) of the magnetic flux guides 136 and 138 are sufficiently high. Moreover, the asymmetry between the two tip ends may minimize the bowing of the first magnetic field 128 away from the two tips 142 and 146 at the edges of the field 128. For example and without limitation, w₁ and w₂ may be 100-300% and 50-150% of the inner diameter of the conduit 122, respectively. However, the present invention may also be practiced using two tip ends having substantially the same width.

The first magnetic field 128 may alternatively be generated by other first magnetic assemblies having different magnetic flux sources and/or magnetic flux guides with other geometric configurations. For example and without limitation, FIG. 10 is a cross-sectional view of another first magnetic assembly 148 for generating the first magnetic field 128 in the upstream section of the conduit 122. The first magnetic assembly 148 includes a magnetic flux source, which comprises first and second permanent magnets 150 and 152, and first and second magnetic flux guides 154 and 156 for conducting the magnetic flux and shaping the first magnetic field 128. The conduit 122, the permanent magnets 150 and 152, and the first and second magnetic flux guides 154 and 156 extend in a direction perpendicular to the drawing.

With continued reference to FIG. 10, the first magnetic flux guide 154 has a first base 158 that may collect magnetic flux and a first tip 160 that may emit magnetic field. The second magnetic flux guide 156 has a second base 162 that may collect magnetic flux and a second tip 164 that may emit magnetic field. The first and second magnetic flux guides 154 and 156 are substantially straight with their tips 160 and 164 pointed toward each other. The first base 158 is physically and/or magnetically coupled to the first permanent magnet 150 at its first pole (e.g., North pole) for conducting the magnetic flux from the first permanent magnet 150. The second base 162 is physically and/or magnetically coupled to the second permanent magnet 152 at its second pole (e.g., South pole) that is opposite to the first pole for conducting the magnetic flux from the second permanent magnet 152. This arrangement of the permanent magnets 150 and 152 renders the first and second tips 160 and 164 having opposite magnetic polarities for generating the first magnetic field 128. The first magnetic flux guide 154 has a smaller cross section, which may be defined herein as the cross-sectional area perpendicular to the flux flow, at the first tip 160 than the first base 158 in order to concentrate the magnetic flux collected from the first permanent magnet 150 to the first tip 160. Similarly, the second tip 164 has a smaller cross section than the second base 162 to concentrate the magnetic flux collected from the second permanent magnet 152 to the second tip 164. Accordingly, each of the tips 160 and 164 has a higher magnetic flux density than the corresponding base 158 or 162. The first and second tips 160 and 164 emit and shape the first magnetic field 128 in the gap therebetween from the concentrated magnetic flux. The geometric configurations of the first and second tips 160 and 164 and the placement of the conduit 122 therebetween may be substantially similar to that of the first magnetic assembly 132 shown in FIG. 9 and described above.

Another example of the first magnetic assembly that is suitable for generating the first magnetic field 128 is shown in FIG. 11. The first magnetic assembly 166 combines the magnetic field guides 136 and 138 of the first magnetic assembly 132 shown in FIG. 9 with the magnetic flux source of the first magnetic assembly 148 shown in FIG. 10. The first base 140 of the first magnetic flux guide 136 is physically and/or magnetically coupled to the first permanent magnet 150 at its first pole (e.g., North pole) for conducting the magnetic flux from the first permanent magnet 150. The second base 144 of the second magnetic flux guide 138 is physically and/or magnetically coupled to the second permanent magnet 152 at its second pole (e.g., South pole) that is opposite to the first pole for conducting the magnetic flux from the second permanent magnet 152. This arrangement of the permanent magnets 150 and 152 renders the first and second tips 142 and 146 having opposite magnetic polarities for generating the first magnetic field 128. The magnetic device 166 may further include an optional third magnetic flux guide 168 having one end physically and/or magnetically coupled to the first permanent magnet 150 at its second pole (e.g., South pole) and the other end physically and/or magnetically coupled to the second permanent magnet 152 at its first pole (e.g., North pole). The third magnetic flux guide 168 conducts the magnetic flux between the first and second permanent magnets 150 and 152 and provides a direct flux conduction path between the first and second permanent magnets 150 and 152, which may further enhance the field strength of the first magnetic field 128.

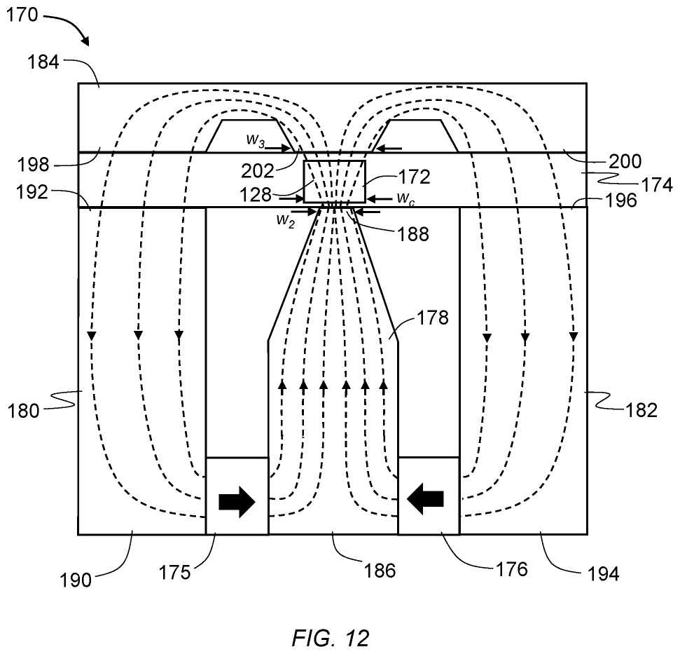

While the above examples show that two pieces of magnetic flux guides may generate the first magnetic field 128, it is worth noting that more than two pieces of magnetic flux guides may be used to generate the first magnetic field 128. FIG. 12 is a cross-sectional view of another example of the first magnetic assembly 170 that is suitable for generating the first magnetic field 128 in a channel 172 embedded in a channel plate 174 for flowing a sample fluid instead of the conduit 122. The first magnetic assembly 170, which generates a first magnetic field 128 in the upstream section of the channel 172, includes a center magnetic flux guide 178 for conducting the magnetic flux and shaping the magnetic field 128, first and second side magnetic flux guides 180 and 182 disposed on opposite sides of the center magnetic flux guide 178 for conducting the magnetic flux, a magnetic flux source, which comprises a first permanent magnet 175 disposed between the center magnetic flux guide 178 and the first side magnetic flux guide 180 and a second permanent 176 disposed between the center magnetic flux guide 178 and the second side magnetic flux guide 182, and a top magnetic flux guide 184 operably disposed on top of the channel plate 174, which is operably disposed on top of the magnetic flux guides 178-182. The channel 172, the channel plate 174, the permanent magnets 175 and 176, and the magnetic flux guides 178-184 extend in a direction perpendicular to the drawing.

With continued reference to FIG. 12, the center magnetic flux guide 178 has a center base 186 that may collect magnetic flux and a center tip 188 that may emit magnetic field. The first side magnetic flux guide 180 has a first base 190 that may collect magnetic flux and a first tip 192 that may emit magnetic field. The first base 190 and the first tip 192 may have substantially the same cross section. The second side magnetic flux guide 182 has a second base 194 that may collect magnetic flux and a second tip 196 that may emit magnetic field. The second base 194 and the second tip 196 may have substantially the same cross section. The center magnetic flux guide 178 and the first and second side magnetic flux guides 180 and 182 may be substantially parallel to each other. The top magnetic flux guide 184 includes first and second top bases 198 and 200 and a top center tip 202. The top center tip 202 and the center tip 188 may be pointed toward each other. The center base 186 is physically and/or magnetically coupled to the first and second permanent magnets 175 and 176 at their first pole (e.g., North pole) for conducting the magnetic flux from the first and second permanent magnets 175 and 176. The first base 190 is physically and/or magnetically coupled to the first permanent magnet 175 at its second pole (e.g., South pole) for conducting the magnetic flux from the first permanent magnet 175. The second base 194 is physically and/or magnetically coupled to the second permanent magnet 176 at its second pole (e.g., South pole) for conducting the magnetic flux from the second permanent magnet 176. The first and second top bases 198 and 200 are magnetically coupled to the first and second tips 192 and 196 through the magnetic fields across the nonmagnetic channel plate 174, respectively. The magnetic flux collected at the first and second top bases 198 and 200 is combined at the top center tip 202. Accordingly, opposite magnetic polarities are respectively formed at the top center tip 202 and the center tip 188 to produce the first magnetic field 128 in the channel 172.

The center magnetic flux guide 178 has a smaller cross section, which may be defined herein as the cross-sectional area perpendicular to the flux flow, at the center tip 188 than the center base 186 in order to concentrate the magnetic flux collected from the first and second permanent magnets 175 and 176 to the center tip 188, resulting in the center tip 188 having a higher magnetic flux density than the center base 188. The top magnetic flux guide 184 may have a smaller cross section at the top center tip 202 than the first and second top bases 198 and 200 in order to concentrate the magnetic flux to the top center tip 202. Moreover, the magnetic flux in the top center tip 202 is collected from both the first and second top bases 198 and 200, further resulting in the top center tip 202 having a higher magnetic flux density than the first and second top bases 198 and 200. The center tip 188 and the top center tip 202 with opposite magnetic polarities emit and shape the first magnetic field 128 in the gap therebetween from the concentrated magnetic flux.

The first magnetic field 128 may be shaped by the tip geometry of the center magnetic flux guide 178 and the top magnetic flux guide 184. Each of the center tip 188 and the top center tip 202 may have a tapering shape with a blunt or flat end. The flat ends of the tips 188 and 202 may be substantially parallel to each other with the channel 172 operably disposed between the two flat ends. The channel plate 174 may be in contact with or in close proximity to the flat ends of the tips 188 and 202 during the cell sorting process. The width of one tip end (e.g., the top center tip 202, wi) may be wider than the width of the channel 172 (w_{c}) while the width of the other tip end (e.g., the center tip, w2) may be comparable to or smaller than w_{c}. For example and without limitation, w₁ and w₂ may be 100-300% and 50-150% of the width of the channel 172, respectively. This tip end geometric configurations may expose most or all of the channel 172 to a magnetic field with high field strength provided that the base-to-tip cross section ratios (i.e., flux concentration factor) of the magnetic flux guides 178 and 184 are sufficiently high. Moreover, the asymmetry between the two tip ends 188 and 202 may minimize the bowing of the first magnetic field 128 away from the two tips 188 and 202 at the edges of the field 128. However, the present invention may also be practiced using two tip ends having substantially the same width.

It is worth noting that the first magnetic assemblies 132, 148, and 166 shown in FIGS. 9-11 may also accommodate the channel plate 174 with the channel 172 embedded therein. The channel plate 174 may be inserted vertically between the two tips with the channel 172 aligned to the two tips that produce the first magnetic field 128.

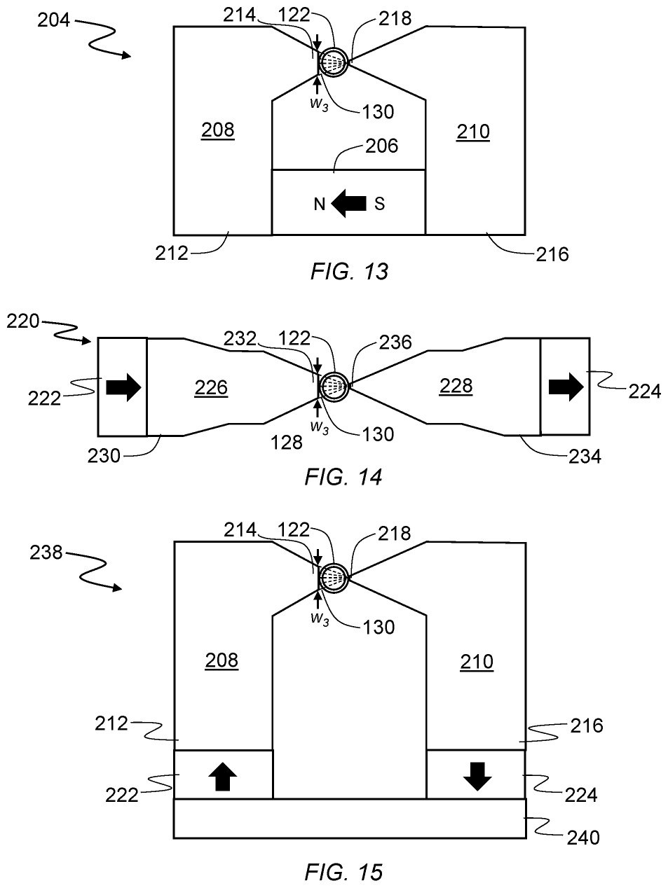

FIG. 13 is a cross-sectional view of a second magnetic assembly 204 in accordance with an embodiment of the present invention. The second magnetic assembly 204 may resemble the first magnetic assembly 132 shown in FIG. 9, except for the tip geometry of the magnetic flux guides. The second magnetic assembly 204, which generates a second magnetic field 130 in the downstream section of the conduit 122, includes a magnetic flux source, which comprises a permanent magnet 206, and first and second magnetic flux guides 208 and 210 for conducting the magnetic flux and shaping the magnetic field 130. In addition to the conduit 122, the permanent magnet 206, and the first and second magnetic flux guides 208 and 210 extend in a direction perpendicular to the drawing and may be attached to a first magnetic assembly, such as but not limited to any one of the first magnetic assemblies shown in FIGS. 9-12.

The first magnetic flux guide 208 has a first base 212 that may collect magnetic flux and a first tip 214 that may emit magnetic field. The second magnetic flux guide 210 has a second base 216 that may collect magnetic flux and a second tip 218 that may emit magnetic field. The first and second magnetic flux guides 208 and 210 are bent with their tips 214 and 218 pointed toward each other. The first and second bases 212 and 216 are physically and/or magnetically coupled to the permanent magnet 206 at its two poles (e.g., North and South poles), respectively, for conducting the magnetic flux from the permanent magnet 206, thereby generating opposite magnetic polarities on the first and second tips 214 and 218, respectively. The first magnetic flux guide 208 has a smaller cross section, which may be defined herein as the cross-sectional area perpendicular to the flux flow, at the first tip 214 than the first base 212 in order to concentrate the magnetic flux collected from the permanent magnet 206 to the first tip 214. Similarly, the second tip 218 has a smaller cross section than the second base 216 to concentrate the magnetic flux collected from the permanent magnet 206 to the second tip 218. Accordingly, each of the tips 214 and 218 has a higher magnetic flux density than the corresponding base 212 or 216. The first and second tips 214 and 218 emit and shape the second magnetic field 130 in the gap therebetween from the concentrated magnetic flux.

The second magnetic field 130 is shaped by the tip geometry of the magnetic flux guides 208 and 210. One of the first and second tips 214 and 218 (e.g., the first tip 214) may have a tapering shape with a blunt or flat end, while the other one (e.g., the second tip 218) may have a tapering shape with a substantially pointed end, especially when compared to the flat end of the first tip 214 and the flat ends of the tips 142/146/160/164/188/202 in the first magnetic assembly. The conduit 122 may be operably disposed between the first and second tips 214 and 218 with the second tip end pointed toward the center of the conduit 122. The conduit 122 may be in contact with or in close proximity to the flat end of first tip 214 and the pointed end of the second tip 218 during the cell sorting process. The width of the tip with the flat end (e.g., the first tip 214, w₃) may be comparable to or narrower than the inner diameter of the conduit 122 while the sharpness of the other tip with pointed end (e.g., the second tip 218) may be limited by the fabrication process. For example and without limitation, w₃ may be 20-150% of the inner diameter of the conduit 122 and the width of the pointed end may be less than 30% of the inner diameter of the conduit 122. In contrast to the tip end geometric configurations of the first magnetic assemblies 132/148/166/170 for generating the first magnetic field 128, this tip end geometric configurations may expose the interior of the conduit 122 to a magnetic field with extremely high local field strength, especially in the immediate vicinity of the pointed tip end where the magnetic flux is highly concentrated. The magnetic field strength, however, rapidly drops off away from the pointed tip end, thereby giving the second magnetic field 130 a high magnetic field gradient.

In an embodiment, the first magnetic flux guide 208 of the second magnetic assembly 204 and the first magnetic flux guide 136 of the first magnetic assembly 132 with different tip shapes are formed from a same (i.e., monolithic) piece of magnetic material; and/or the second magnetic flux guide 210 of the second magnetic assembly 204 and the second magnetic flux guide 138 of the first magnetic assembly 132 with different tip shapes are formed from a same piece of magnetic material; and/or the permanent magnet 206 of the second magnetic assembly 204 and the permanent magnet 134 of the first magnetic assembly 132 are formed from a same (i.e., monolithic) piece of magnetic material. Alternatively, the first magnetic flux guide 208 may have a pointed tip end and may be formed with the first magnetic flux guide 136 from a same piece of magnetic material, and the second magnetic flux guide 210 may have a flat tip end and may be formed with the second magnetic flux guide 138 from a same piece of magnetic material.

FIG. 14 is a cross-sectional view of another second magnetic assembly 220 for generating a second magnetic field 130 in the downstream section of the conduit 122. The second magnetic assembly 220 may resemble the first magnetic assembly 148 shown in FIG. 10, except for the tip geometry of the magnetic flux guides. The second magnetic assembly 220 includes a magnetic flux source, which comprises first and second permanent magnets 222 and 224, and first and second magnetic flux guides 226 and 228 for conducting the magnetic flux and shaping the second magnetic field 130. In addition to the conduit 122, the permanent magnets 222 and 224, and the first and second magnetic flux guides 226 and 228 may extend in a direction perpendicular to the drawing and may be attached to a first magnetic assembly, such as but not limited to any one of the first magnetic assemblies shown in FIGS. 9-12.

With continued reference to FIG. 14, the first magnetic flux guide 226 has a first base 230 that may collect magnetic flux and a first tip 232 that may emit magnetic field. The second magnetic flux guide 228 has a second base 234 that may collect magnetic flux and a second tip 236 that may emit magnetic field. The first and second magnetic flux guides 226 and 228 are substantially straight with their tips 232 and 236 pointed toward each other. The first base 230 is physically and/or magnetically coupled to the first permanent magnet 222 at its first pole (e.g., North pole) for conducting the magnetic flux from the first permanent magnet 222. The second base 234 is physically and/or magnetically coupled to the second permanent magnet 224 at its second pole (e.g., South pole) that is opposite to the first pole for conducting the magnetic flux from the second permanent magnet 224. This arrangement of the permanent magnets 222 and 224 renders the first and second tips 232 and 236 having opposite magnetic polarities for generating the second magnetic field 130. The first magnetic flux guide 226 has a smaller cross section, which may be defined herein as the cross-sectional area perpendicular to the flux flow, at the first tip 232 than the first base 230 in order to concentrate the magnetic flux collected from the first permanent magnet 222 to the first tip 232. Similarly, the second tip 236 has a smaller cross section than the second base 234 to concentrate the magnetic flux collected from the second permanent magnet 224 to the second tip 236. Accordingly, each of the tips 232 and 236 has a higher magnetic flux density than the corresponding base 230 or 234. The first and second tips 232 and 236 emit and shape the second magnetic field 130 in the gap therebetween from the concentrated magnetic flux. The geometric configurations of the first and second tips 232 and 236 and the placement of the conduit 122 therebetween may be substantially similar to that of the second magnetic assembly 204 shown in FIG. 13 and described above.

In an embodiment, the first magnetic flux guide 226 of the second magnetic assembly 220 and the first magnetic flux guide 154 of the first magnetic assembly 148 with different tip shapes are formed from a same (i.e., monolithic) piece of magnetic material; and/or the second magnetic flux guide 228 of the second magnetic assembly 220 and the second magnetic flux guide 156 of the first magnetic assembly 148 with different tip shapes are formed from a same piece of magnetic material; and/or the first permanent magnet 222 of the second magnetic assembly 220 and the first permanent magnet 150 of the first magnetic assembly 148 are formed from a same (i.e., monolithic) piece of magnetic material; and/or the second permanent magnet 224 of the second magnetic assembly 220 and the second permanent magnet 152 of the first magnetic assembly 148 are formed from a same piece of magnetic material. Alternatively, the first magnetic flux guide 226 may have a pointed tip end and may be formed with the first magnetic flux guide 154 from a same piece of magnetic material, and the second magnetic flux guide 228 may have a flat tip end and may be formed with the second magnetic flux guide 156 from a same piece of magnetic material.

FIG. 15 is a cross-sectional view of still another second magnetic assembly 238 for generating the second magnetic field 130 in the downstream section of the conduit 122. The second magnetic assembly 238 may resemble the first magnetic assembly 166 shown in FIG. 11, except for the tip geometry of the magnetic flux guides. The second magnetic assembly 238 combines the magnetic field guides 208 and 210 of the second magnetic assembly 204 shown in FIG. 13 with the magnetic flux source of the second magnetic assembly 220 shown in FIG. 14. The first base 212 of the first magnetic flux guide 208 is physically and/or magnetically coupled to the first permanent magnet 222 at its first pole (e.g., North pole) for conducting the magnetic flux from the first permanent magnet 222. The second base 216 of the second magnetic flux guide 210 is physically and/or magnetically coupled to the second permanent magnet 224 at its second pole (e.g., South pole) that is opposite to the first pole for conducting the magnetic flux from the second permanent magnet 224. This arrangement of the permanent magnets 222 and 224 renders the first and second tips 214 and 218 having opposite magnetic polarities for generating the second magnetic field 130. The second magnetic assembly 238 may further include an optional third magnetic flux guide 240 having one end physically and/or magnetically coupled to the first permanent magnet 222 at its second pole (e.g., South pole) and the other end physically and/or magnetically coupled to the second permanent magnet 224 at its first pole (e.g., North pole). The third magnetic flux guide 240 conducts the magnetic flux between the first and second permanent magnets 222 and 224 and provides a direct flux conduction path between the first and second permanent magnets 222 and 224, which may further enhance the field strength and/or field gradient of the second magnetic field 130.

In addition to the conduit 122, the permanent magnets 222 and 224, and the first, second, and third magnetic flux guides 208, 210, and 240 may extend in a direction perpendicular to the drawing and may be attached to a first magnetic assembly, such as but not limited to any one of the first magnetic assemblies shown in FIGS. 9-12.

In an embodiment, the first magnetic flux guide 208 of the second magnetic assembly 238 and the first magnetic flux guide 136 of the first magnetic assembly 166 with different tip shapes are formed from a same (i.e., monolithic) piece of magnetic material; and/or the second magnetic flux guide 210 of the second magnetic assembly 238 and the second magnetic flux guide 138 of the first magnetic assembly 166 with different tip shapes are formed from a same piece of magnetic material; and/or the third magnetic flux guide 240 of the second magnetic assembly 238 and the third magnetic flux guide 168 of the first magnetic assembly 166 are formed from a same piece of magnetic material; and/or the first permanent magnet 222 of the second magnetic assembly 238 and the first permanent magnet 150 of the first magnetic assembly 166 are formed from a same (i.e., monolithic) piece of magnetic material; and/or the second permanent magnet 224 of the second magnetic assembly 238 and the second permanent magnet 152 of the first magnetic assembly 166 are formed from a same piece of magnetic material. Alternatively, the first magnetic flux guide 208 may have a pointed tip end and may be formed with the first magnetic flux guide 136 from a same piece of magnetic material, and the second magnetic flux guide 210 may have a flat tip end and may be formed with the second magnetic flux guide 138 from a same piece of magnetic material.

Like the first magnetic assemblies 132, 148, and 166 shown in FIGS. 9-11, the second magnetic assemblies 204, 220, and 238 may also accommodate the channel plate 174 with the channel 172 embedded therein. The channel plate 174 may be inserted vertically between the two tips with the channel 172 aligned to the two tips that produce the second magnetic field 130.

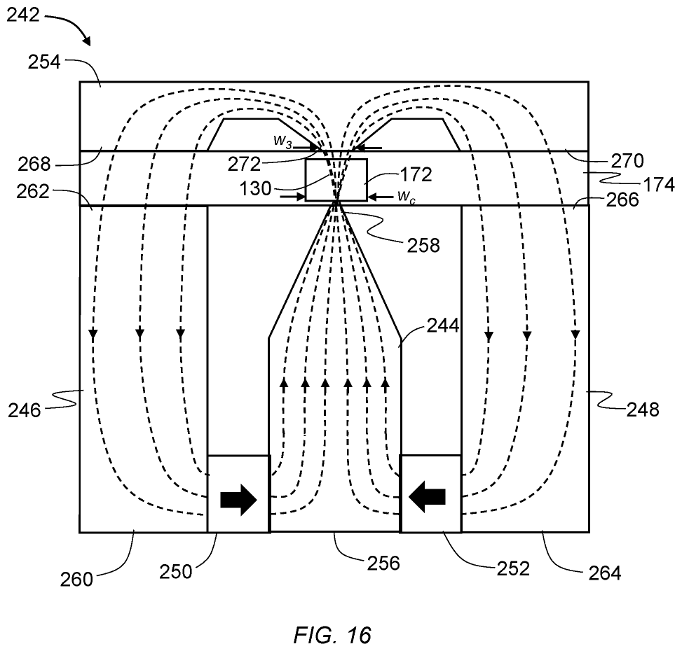

FIG. 16 is a cross-sectional view of another example of the second magnetic assembly 242 that is suitable for generating the second magnetic field 130 in a channel 172 embedded in a channel plate 174 for flowing a sample fluid instead of the conduit 122. The second magnetic assembly 242 may resemble the first magnetic assembly 170 shown in FIG. 12, except for the tip geometry of the top and center magnetic flux guides. The second magnetic assembly 242, which generates the second magnetic field 130 in the downstream section of the channel 172, includes a center magnetic flux guide 244 for conducting the magnetic flux and shaping the second magnetic field 130, first and second side magnetic flux guides 246 and 248 disposed on opposite sides of the center magnetic flux guide 244 for conducting the magnetic flux, a magnetic flux source, which comprises a first permanent magnet 250 disposed between the center magnetic flux guide 244 and the first side magnetic flux guide 246 and a second permanent 252 disposed between the center magnetic flux guide 244 and the second side magnetic flux guide 248, and a top magnetic flux guide 254 operably disposed on top of the channel plate 174, which is operably disposed on top of the magnetic flux guides 244-248. In addition to the channel 172 and the channel plate 174, the permanent magnets 250 and 252 and the magnetic flux guides 244-248 and 254 may extend in a direction perpendicular to the drawing and may be attached to a first magnetic assembly, such as but not limited to any one of the first magnetic assemblies shown in FIGS. 9-12.

With continued reference to FIG. 16, the center magnetic flux guide 244 has a center base 256 that may collect magnetic flux and a center tip 258 that may emit magnetic field. The first side magnetic flux guide 246 has a first base 260 that may collect magnetic flux and a first tip 262 that may emit magnetic field. The second side magnetic flux guide 248 has a second base 264 that may collect magnetic flux and a second tip 266 that may emit magnetic field. The center magnetic flux guide 244 and the first and second side magnetic flux guides 246 and 248 may be substantially parallel to each other. The top magnetic flux guide 254 includes first and second top bases 268 and 270 and a top center tip 272. The top center tip 272 and the center tip 258 are pointed toward each other. The center base 256 is physically and/or magnetically coupled to the first and second permanent magnet 250 and 252 at their first pole (e.g., North pole) for conducting the magnetic flux from the first and second permanent magnets 250 and 252. The first base 260 is physically and/or magnetically coupled to the first permanent magnet 250 at its second pole (e.g., South pole) for conducting the magnetic flux from the first permanent magnet 250. The second base 264 is physically and/or magnetically coupled to the second permanent magnet 252 at its second pole (e.g., South pole) for conducting the magnetic flux from the second permanent magnet 252. The first and second top bases 268 and 270 are magnetically coupled to the first and second tips 262 and 266 through the magnetic fields across the nonmagnetic channel plate 174, respectively. The magnetic flux collected at the first and second top bases 268 and 270 is combined at the top center tip 272. Accordingly, opposite magnetic polarities are respectively formed at the top center tip 272 and the center tip 258 to produce the second magnetic field 130 in the channel 172.

The center magnetic flux guide 244 has a smaller cross section, which may be defined herein as the cross-sectional area perpendicular to the flux flow, at the center tip 258 than the center base 256 in order to concentrate the magnetic flux collected from the first and second permanent magnets 250 and 252 to the center tip 258, resulting in the center tip 258 having a higher magnetic flux density than the center base 256. The top magnetic flux guide 254 may have a smaller cross section at the top center tip 272 than the first and second top bases 268 and 270 in order to concentrate the magnetic flux to the top center tip 272. Moreover, the magnetic flux in the top center tip 272 is collected from both the first and second top bases 268 and 270, further resulting in the top center tip 272 having a higher magnetic flux density than the first and second top bases 268 and 270. The center tip 258 and the top center tip 272 with opposite magnetic polarities emit and shape the second magnetic field 130 in the gap therebetween from the concentrated magnetic flux.

The second magnetic field 130 may be shaped by the tip geometry of the magnetic flux guides 244 and 254. One of the center tip 258 and the top center tip 272 (e.g., the top center tip 272) may have a tapering shape with a blunt or flat end, while the other one (e.g., the center tip 258) may have a tapering shape with a substantially pointed end, especially when compared to the flat end of the top center tip 272 and/or the flat ends of the tips 142/146/160/164/188/202 in the first magnetic assembly and/or the flat ends of the tips 214/232 in the second magnetic assembly. The channel 172 and the channel plate 174 may be operably disposed between the center tip 258 and the top center tip 272 with their respective tip ends aligned to the center of the channel 172. The channel plate 174 may be in contact with or in close proximity to the flat end of the top center tip 272 and the pointed end of the center tip 258 during the cell sorting process. The width of the tip with the flat end (e.g., the top center tip 272, w3) may be comparable to or narrower than the width of the channel 172 (w_{c}) while the sharpness of the other tip with pointed end (e.g., the center tip 258) may be limited by the fabrication process. For example and without limitation, w₃ may be 20-150% of the width of the channel 172 and the width of the pointed end may be less than 30% of the width of the channel 172. In contrast to the tip end geometric configurations of the first magnetic assemblies 132/148/166/170 for generating the first magnetic field 128, this tip end geometric configurations may expose the channel 172 to a magnetic field with extremely high local field strength, especially in the immediate vicinity of the pointed tip end where the magnetic flux is highly concentrated. The magnetic field strength, however, rapidly drops off away from the pointed tip end, thereby giving the second magnetic field 130 a high magnetic field gradient.

In an embodiment, the center magnetic flux guide 244 of the second magnetic assembly 242 and the center magnetic flux guide 178 of the first magnetic assembly 170 with different tip shapes are formed from a same (i.e., monolithic) piece of magnetic material; and/or the first side magnetic flux guide 246 of the second magnetic assembly 242 and the first side magnetic flux guide 180 of the first magnetic assembly 170 are formed from a same piece of magnetic material; and/or the second side magnetic flux guide 248 of the second magnetic assembly 242 and the second side magnetic flux guide 182 of the first magnetic assembly 170 are formed from a same piece of magnetic material; and/or the top magnetic flux guide 254 of the second magnetic assembly 242 and the top magnetic flux guide 184 of the first magnetic assembly 170 with different tip shapes are formed from a same piece of magnetic material; and/or the first permanent magnet 250 of the second magnetic assembly 242 and the first permanent magnet 175 of the first magnetic assembly 170 are formed from a same (i.e., monolithic) piece of magnetic material; and/or the second permanent magnet 252 of the second magnetic assembly 242 and the second permanent magnet 176 of the first magnetic assembly 170 are formed from a same piece of magnetic material. Alternatively, the top magnetic flux guide 254 may have a pointed tip end and may be formed with the top magnetic flux guide 184 from a same piece of magnetic material, and the center magnetic flux guide 244 may have a flat tip end and may be formed with the center magnetic flux guide 178 from a same piece of magnetic material.

The conduit 122 is nonmagnetic and may be made of a rigid material, such as but not limited to glass, metal, or ceramics, or a flexible or pliable material, such as but not limited to rubber, plastic, or other polymeric materials. The conduit 122 may be operable to be pressed and deformed against the tip ends, thereby allowing the sample fluid to flow in closer proximity to the tips emitting the magnetic field. The channel plate 174 may be nonmagnetic and may be made of a rigid material, such as but not limited to glass, metal, ceramic, silicon, or any suitable polymeric material.

The magnetic flux guides 136/138/154/156/168/178-184/208/210/226/228/240/244-248/254 each may be made of a soft magnetic material or a material with relatively high magnetic permeability that comprises any one of iron (Fe), cobalt (Co), nickel (Ni), or any combination thereof. For example and without limitation, any of the magnetic flux guides may be made of a permalloy comprising nickel and iron.

While the present invention has been shown and described with reference to certain preferred embodiments, it is to be understood that those skilled in the art will no doubt devise certain alterations and modifications thereto which nevertheless include the true spirit and scope of the present invention. Thus, the scope of the invention should be determined by the appended claims and their legal equivalents, rather than by examples given.

Any element in a claim that does not explicitly state “means for” performing a specified function, or “step for” performing a specific function, is not to be interpreted as a “means” or “step” clause as specified in 35 U.S.C. § 112, ¶ 6. In particular, the use of “step of” in the claims herein is not intended to invoke the provisions of 35 U.S.C. § 112, ¶6.

What is claimed is:

1. A method for sorting biological objects comprising the steps of:
providing a magnetic device that includes a conduit or channel having upstream and downstream sections and a magnetic means for generating first and second magnetic fields in the upstream and downstream sections, respectively;
flowing a sample fluid that includes magnetically labeled biological objects and unlabeled biological objects through the upstream section to magnetically saturate the magnetically labeled biological objects by the first magnetic field; and
flowing the sample fluid from the upstream section continuously to the downstream section to collect the magnetically labeled biological objects on a wall of the downstream section by the second magnetic field,
wherein the first magnetic field in the upstream section has a higher average field strength than the second magnetic field in the downstream section.

2. The method of claim 1, wherein the second magnetic field in the downstream section has a higher field gradient than the first magnetic field in the upstream section.

3. A magnetic device for sorting biological objects comprising:
a first magnetic assembly including:
a first magnetic flux source;
a first magnetic flux guide having a first base and a first tip with a first blunt end; and
a second magnetic flux guide having a second base and a second tip with a second blunt end facing the first tip,
wherein the first and second bases are magnetically coupled to the first magnetic flux source to generate opposite magnetic polarities on the first and second tips, respectively, thereby producing a first magnetic field between the first and second tips;
a second magnetic assembly disposed adjacent to the first magnetic assembly and including:
a second magnetic flux source;
a third magnetic flux guide having a third base and a third tip with a third blunt end; and
a fourth magnetic flux guide having a fourth base and a fourth tip with a substantially pointed end facing the third tip,
wherein the third and fourth bases are magnetically coupled to the second magnetic flux source to generate opposite magnetic polarities on the third and fourth tips, respectively, thereby producing a second magnetic field between the third and fourth tips; and
a conduit operably disposed between the first blunt end of the first tip and the second blunt end of the second tip and between the third blunt end of the third tip and the substantially pointed end of the fourth tip.

4. The magnetic device of claim 3, wherein the first magnetic field has a higher average field strength than the second magnetic field.

5. The magnetic device of claim 3, wherein the second magnetic field has a higher field gradient than the first magnetic field.

6. The magnetic device of claim 3, wherein the first and second magnetic flux sources each comprise one or more permanent magnets.

7. The magnetic device of claim 3, wherein the first and second magnetic flux sources share one or more permanent magnets.

8. The magnetic device of claim 3, wherein the first magnetic flux source comprises a permanent magnet magnetically coupled to the first and second bases at opposite poles of the permanent magnet, respectively.

9. The magnetic device of claim 3, wherein the first magnetic flux source comprises a first permanent magnet magnetically coupled to the first base at a first pole of the first permanent magnet and a second permanent magnet magnetically coupled to the second base at a second pole of the second permanent magnet.

10. The magnetic device of claim 9 further comprising a fifth magnetic flux guide with one end magnetically coupled to the first permanent magnet at a second pole thereof and the other end magnetically coupled to the second permanent magnet at a first pole thereof.

11. The magnetic device of claim 3, wherein the second magnetic flux source comprises a permanent magnet magnetically coupled to the third and fourth bases at opposite poles of the permanent magnet, respectively.

12. The magnetic device of claim 3, wherein the second magnetic flux source comprises a first permanent magnet magnetically coupled to the third base at a first pole of the first permanent magnet and a second permanent magnet magnetically coupled to the fourth base at a second pole of the second permanent magnet.

13. The magnetic device of claim 12 further comprising a fifth magnetic flux guide with one end magnetically coupled to the first permanent magnet at a second pole thereof and the other end magnetically coupled to the second permanent magnet at a first pole thereof.

14. The magnetic device of claim 3, wherein the first base has a larger cross section than the first tip, and the second base has a larger cross section than the second tip.

15. The magnetic device of claim 3, wherein the third base has a larger cross section than the third tip, and the fourth base has a larger cross section than the fourth tip.

16. The magnetic device of claim 3, wherein the first blunt end has a larger cross section than the second blunt end.

17. The magnetic device of claim 16, wherein a width of the first blunt end is greater than an inner diameter of the conduit.

18. The magnetic device of claim 16, wherein a width of the second blunt end is greater than an inner diameter of the conduit.

19. The magnetic device of claim 3, wherein the first, second, third, and fourth magnetic flux guides each comprise a soft magnetic material.

20. The magnetic device of claim 3, wherein the first magnetic flux guide and one of the third and fourth magnetic flux guides are formed from a same piece of soft magnetic material.
